(19) United States
(12) Patent Application Publication
(10) Pub. No.: US 2015/0119742 A1
(43) Pub. Date: Apr. 30, 2015

(54) NASAL OXYGEN MASK AND BREATHING CIRCUIT ASSEMBLY

(71) Applicant: Rutgers, The State University of New Jersey, New Brunswick, NJ (US)

(72) Inventors: James Tse, Manalapan, NJ (US); Rose Alloteh, Monmouth Junction, NJ (US); Shaul Cohen, Livingston, NJ (US); Sylviana Barsoum, East Brunswick, NJ (US)

(21) Appl. No.: 14/527,247

(22) Filed: Oct. 29, 2014

Related U.S. Application Data
(60) Provisional application No. 61/897,556, filed on Oct. 30, 2013.

(51) Int. Cl. A61M 16/08; A61M 16/06; A61B 5/00; A61M 16/20; A61B 5/097; A61B 5/083; A61M 16/04; A61M 16/10

(52) U.S. Cl. CPC A61M 16/085; A61M 16/0493; A61M 16/0622; A61M 16/0672; A61M 16/0683; A61M 16/104; A61M 16/209; A61M 16/1005; A61B 5/097; A61B 5/0836; A61B 5/0833; A61B 5/4836; A61M 2205/3327; A61M 2240/00
USPC 600/531; 128/200.26; 128/206.26; 128/207.13; 128/202.27; 128/205.25; 600/543

(57) ABSTRACT

A nasal mask assembly including a nasal mask with a dome shaped body. The dome shaped body has an inner rim and an outer surface. The inner rim has a rounded triangular shape and a tube connector extends from the outer surface. The inner rim is configured such that a bottom portion thereof sits on or above the patient's upper lip while an upper portion thereof extends across the bridge of the patient's nose.

This application claims the benefit of U.S. Provisional Application No. 61/897,556, filed on Oct. 30, 2013, the contents of which are incorporated herein by reference.

### FIELD OF THE INVENTION

This invention relates to a surgical procedure nasal oxygen mask. More particularly, the invention relates to a nasal oxygen mask that provides a continuous positive airway pressure (CPAP) mask while maintaining the patient's mouth clear for a surgical procedure.

### BACKGROUND OF THE INVENTION

Patients undergoing upper GI endoscopy (EGD) routinely receive intravenous sedation and oxygen (O₂) via nasal cannula. Nasal cannula becomes ineffective in delivering O₂ when the patient's mouth is kept open by a bite-block for endoscope. Over-sedation and/or airway obstruction may cause severe hypoxemia (O₂ desaturation) and the procedure has to be interrupted and the endoscope has to be withdrawn in order to perform assisted ventilation and oxygenation through a face mask or an endrotracheal tube. Obese patients have increased risk of respiratory complications during sedation due to a myriad of factors, such as narrow nasal airway, redundant soft tissue in oropharynx, obstructive sleep apnea (OSA), decreased lung capacity and increased O₂ consumption. As gastric bypass, stapling and banding procedures have recently gained popularity; the number of pre-procedure screening and post-procedure interventional EGDs is increasing. Anesthesiologists and GI endoscopists are facing more challenging cases than before.

Obese patients with OSA have an easily collapsible nasal airway (velopharynx) during sleep and under sedation. They may require home nasal or face mask which provides continuous positive airway pressure (CPAP) to prevent airway obstruction during sleep. While under sedation, they may require frequent chin-lift, jaw-thrust and/or using nasopharyngeal trumpets to keep their airway open. Inserting a PVC nasopharyngeal trumpet may cause severe nose bleed.

### SUMMARY OF THE INVENTION

In at least one aspect, the present invention provides a nasal mask assembly including a nasal mask with a dome shaped body. The dome shaped body has an inner rim and an outer surface. The inner rim has a rounded triangular shape and a tube connector extends from the outer surface. The inner rim is configured such that a bottom portion thereof sits on or above the patient's upper lip while an upper portion thereof extends across the bridge of the patient's nose. An inflatable chamber may extend about the inner rim with a sealed inflation port proximate the bottom portion of the rim. A connection ring with an annular body and a plurality of connection pegs is positioned about the tubing connector. A strap assembly includes a plurality of straps configured to engage the connection pegs and maintain the nasal mask against a patient's face about the patient's nose. A bite-block may be positioned in the patient's mouth without interference from the nasal mask assembly. A nasal cannula may be secured proximate to the patient's uncovered mouth and is configured to intake CO₂ and O₂ proximate to the patient's mouth. A sampling line of the nasal mask and the nasal cannula may be connected to a monitoring system to monitor the CO₂ and O₂ level.

### BRIEF DESCRIPTION OF THE DRAWINGS

The accompanying drawings, which are incorporated herein and constitute part of this specification, illustrate the presently preferred embodiments of the invention, and, together with the general description given above and the detailed description given below, serve to explain the features of the invention. In the drawings:

FIG. 1 is a front plan view of nasal mask assembly in accordance with an exemplary embodiment of the present invention.

FIG. 2 is a side elevation view of the nasal mask assembly of FIG. 1.

FIG. 3 is a top plan view of the nasal mask assembly of FIG. 1.

FIG. 4 is a top plan view similar to FIG. 3 with the connection ring separated from the nasal mask.

FIG. 5 is a perspective view of an exemplary strap assembly for use with the nasal mask assembly of FIG. 1.

FIG. 6 illustrates an exemplary nasal mask assembly positioned on a patient with an exemplary strap assembly.

FIG. 7 illustrates an exemplary nasal mask assembly positioned on a patient during an EGD procedure.

FIG. 8 illustrates the exemplary nasal mask assembly of FIG. 7 with a face tent positioned over the face of the patient.

FIG. 9 is a perspective view illustrating a connector interconnecting components of the nasal mask assembly and the monitoring assembly.

### DETAILED DESCRIPTION OF THE INVENTION

In the drawings, like numerals indicate like elements throughout. Certain terminology is used herein for convenience only and is not to be taken as a limitation on the present invention. The following describes preferred embodiments of the present invention. However, it should be understood, based on this disclosure, that the invention is not limited by the preferred embodiments described herein.

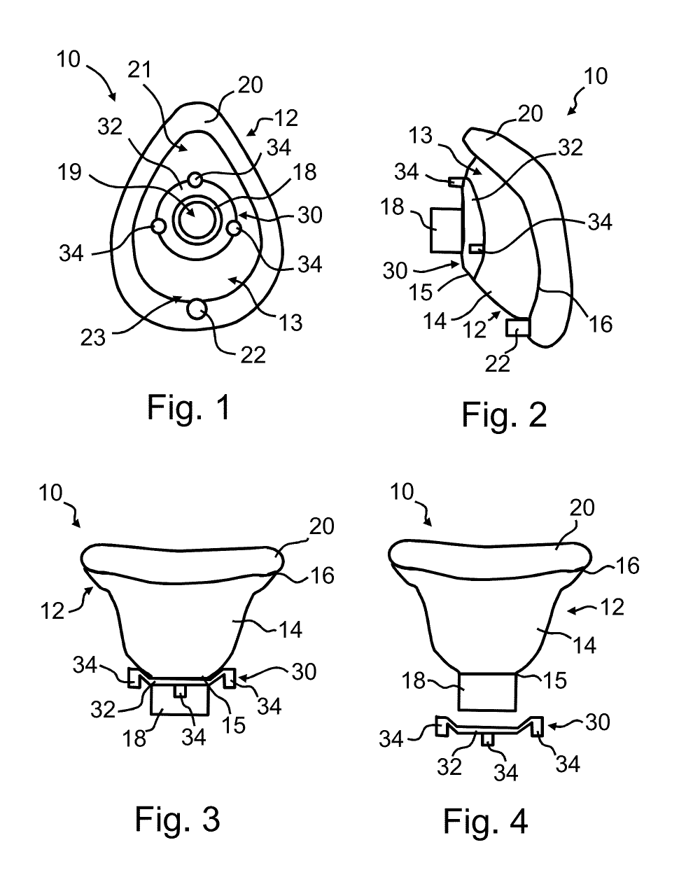

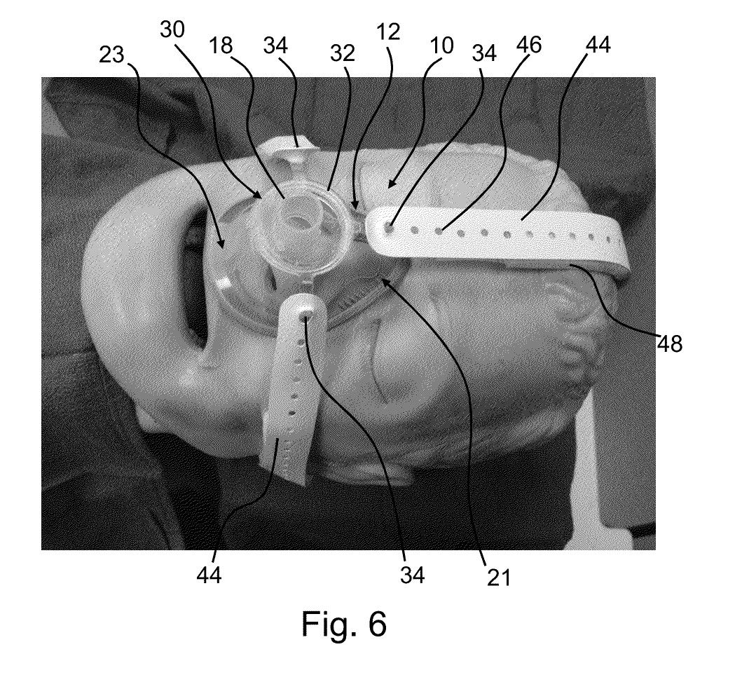

Referring to FIGS. 1-6, an exemplary embodiment of a nasal mask assembly 10 in accordance with an embodiment of the invention will be described. The nasal mask assembly 10 includes a nasal mask 12, a connection ring 30 and a strap assembly 40. The nasal mask 12 includes a dome shaped body 14 which extends from an inner rim 16 to an outer surface 15. A generally hollow chamber 13 is defined within the dome shaped body 14. A tubing connector 18 with an opening 19 into the chamber 13 is provided on the outer surface 15. The dome shaped body 14 is preferably transparent such that the patient's nose within the chamber 13 is visible. The inner rim 16 preferably has a rounded triangular configuration with a narrowed upper end 21 and a wider bottom end 23. The nasal mask 12 is sized such that the bottom end 23 of the mask sits above the patient's upper lip while the upper end 21 is positioned along the bridge of the patient's nose (see FIG. 6). With an adult patient, the nasal mask 14 may take the form of a pediatric sized nose and mouth covering face mask.

In the exemplary embodiment, an inflatable chamber 20 extends about the rim 16 of the dome shaped body 14. A sealed port 22 is in fluid communication with the chamber 20 and facilitates inflation and deflation of the chamber 20. Inflation of the chamber 20 increases sealing of the mask chamber 13 about the patient's nose and also helps to control the positioning of the body 14 relative to the patient's face, i.e. the body 14 may be moved from the face to provide more room for a larger nose by inflating the chamber 20. In the present embodiment, the sealed port 22 is positioned along the bottom end 23 of the mask 12 and thereby avoids interference with the strap assembly as described below.

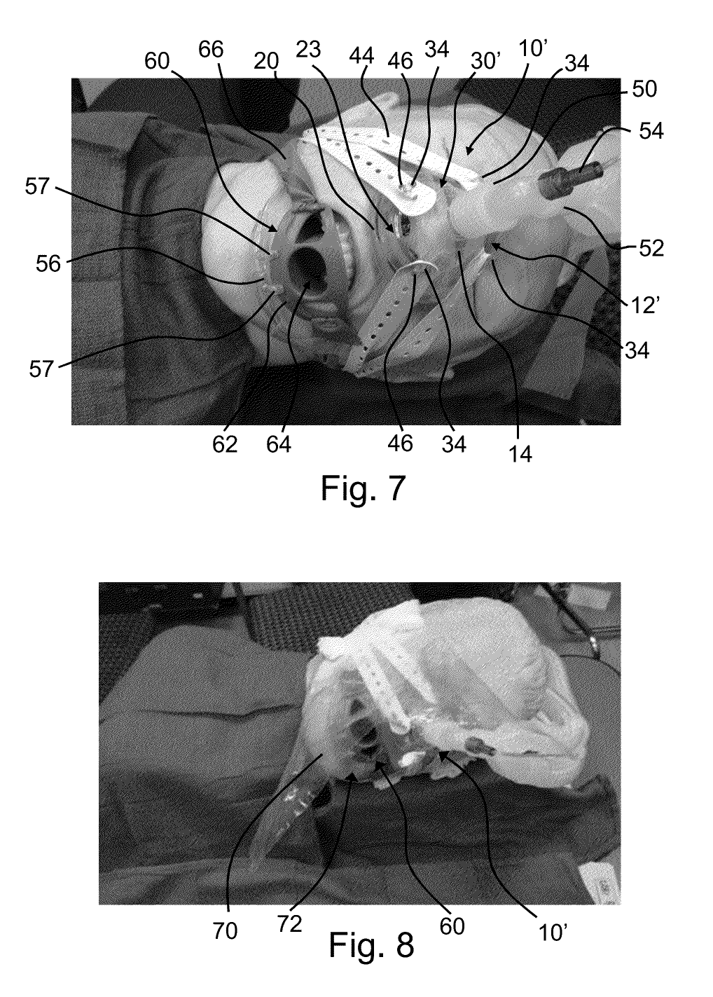

In the exemplary embodiment, the connection ring 30 is formed independent of the nasal mask 12 and is configured to be positioned along the outer surface 15 about the tubing connector 18. The connection ring 30 includes an annular body 32 which complements the shape of the outer surface 15 of the dome shaped body 14. A plurality of connection pegs 34 extend from the annular body 32. In the embodiment illustrated in FIGS. 1-6, the connection ring 30 includes three connection pegs 34 and the strap assembly 40 includes three corresponding straps 40. More or fewer pegs and straps may be utilized. For example, the embodiment of the nasal mask assembly 10′ illustrated in FIGS. 7 and 8 includes a connection ring 30′ with four connection pegs 34 and a strap assembly with four straps 44. The connection ring 30 may be secured to the nasal mask 14, for example, via a friction fit or via tape, or the connection ring 30 may simply be positioned about the tubing connector 18 and secured upon connection of the strap assembly 40. Alternatively, the connection ring 30 may be integrally formed with the nasal mask 12.

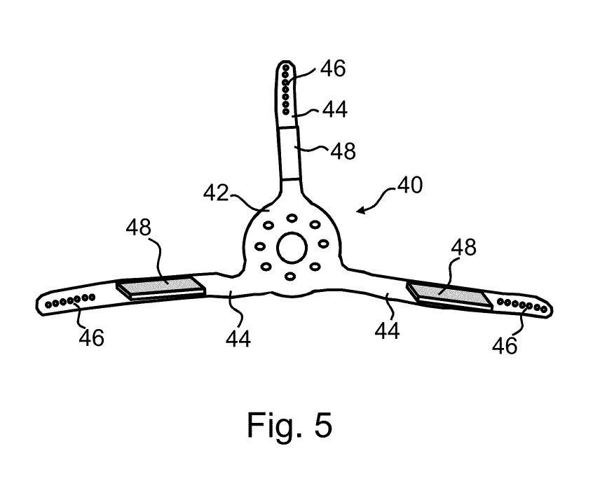

Referring to FIG. 5, the illustrated strap assembly 40 includes a central body 42 with a plurality of connecting straps 44 extending therefrom. In use, the central body 42 is configured to be positioned on the back of the patient's head and each of the straps 44 extended and connected to a respective connection peg 34. Each strap 44 has a plurality of connection holes 46 configured to receive a respective peg 34. The plurality of holes 46 allow the straps 44 to be adjustable. In the illustrated embodiment, each strap 44 includes a cushioning pad 48, for example, foam padding, which helps to minimize discomfort to the patient when the nasal mask assembly 10 is worn.

FIGS. 7 and 8 illustrate an exemplary nasal mask assembly 10′ positioned on a patient and connected to a breathing circuit. The breathing circuit includes tubing 52 connected via a connector 50 to the tubing connector 18. A sampling line 54 preferably extends from the tubing 52. The patient breathes through the nasal mask 14 with the pressure-relief valve of the breathing circuit open. After a few minutes of pre-oxygenation, sedation can be induced in the usual manner. The pressure-relief valve can be adjusted to provide a desired CPAP. This nasal mask can also be used to deliver assisted ventilation if the patient becomes apneic. Inspiratory O₂ concentrations can be adjusted by adding fresh air.

In an EGD procedure, a bite-block 60 is positioned in the mouth of the patient. The bite-block 60 includes a body 62 with a hollow tube 64 extending therefrom and between the upper and lower teeth of the patient. The hollow tube 64 provides a clear path for an endoscope or other surgical tools and the nasal mask assembly 10 does not interfere with such procedure.

A nasal cannula 56 is secured on the lower rim of the bite-block body 62 and includes one or more ports 57 which intake outflow from the patient's mouth. The nasal cannula 56 thereby facilitates CO₂ and O₂ monitoring. As illustrated in FIG. 8, a clean, clear plastic sheet 70 (TSE “mask”) may be placed such that it covers the patient's mouth and nasal cannula and defines an at least partially enclosed area 72. It has bound found that the TSE mask improves CO₂ wave form and the quality of capnography. It may also improve the ability to detect respiratory depression and/or airway obstruction.

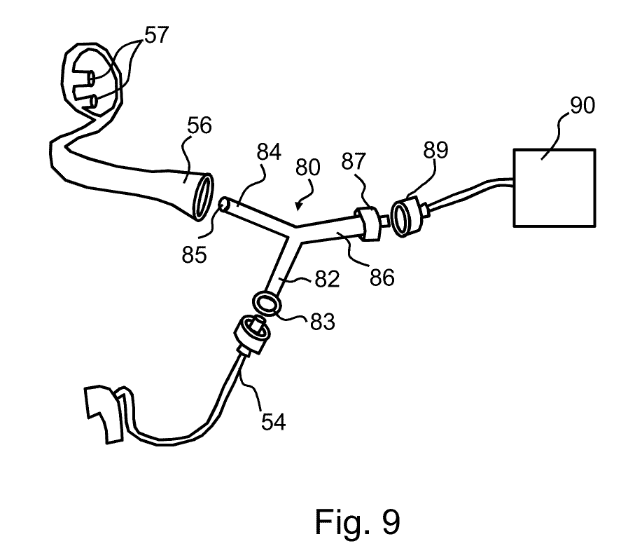

Referring to FIG. 9, a three-way connector 80 interconnects the CO₂ lines of the various elements of the breathing circuit assembly. In the illustrated embodiment, a first leg 82 includes a connector 83 configured for connection to the sampling line 54 while a second leg 84 includes a connector 85 for connection to the nasal cannula 56. The first and second legs 82, 84 are fluidly connected with a third let 86 with a connector 87 configured for connection to a connector 89 of a monitoring system 90 or the like.

The nasal mask assembly 10 and breathing circuit assembly keeps the nasal airway open in deeply sedated obese patients with OSA. It improves oxygenation and prevents severe hypoxemia in these patients. It is simple and easy to use with existing anesthesia machine. It can be used to provide immediate assisted ventilation if the patient becomes apneic without interfering with the procedure. It has the advantages over a face CPAP endoscopy mask because it allows easy access to the oral cavity for suctioning fluid without removing the O₂ supply from the patient. Besides EGD, it can be used for many other procedures that perform under sedation, such as colonoscopy, cystoscopy or breast biopsy. With these procedures, patients usually breathe through their nose and this nasal mask provides CPAP. It can also be used for non-obese patients and patients without OSA. It can also be used for induction of general anesthesia, especially patients with full beard or patients without teeth. Using the existing breathing circuit and the anesthesia machine, it may improve patient safety at a very low cost.

The nasal mask assembly 10 can also be used in obese and non-obese patients under deep sedation for other procedures, such as colonoscopy and upper body procedures (such as breast biopsy, lumpectomy). Fire in the operation room is recently a major concern according to the 2013 Updated Guideline of the American Society of Anesthesiologists. Using the nasal mask assembly 10 can reduce the risk of fire hazard as recommended by the guideline. The fresh O₂ and air flows can be adjusted to deliver the lowest O₂ concentration to achieve good oxygenation. The exhaled O₂ can be removed by the scavenger system to avoid pooling O₂ under surgical drapes near the surgical site. In addition, it covers only the nose and is more comfortable to the patient than a face mask.

The nasal mask assembly 10 can be used in the operating rooms and remote anesthesia locations where anesthesia machines are available. Additional light-weight portable parts and adapters can also be developed, so that this mask/circuit can be attached to any O₂ supply line or O₂ tank and can used in endoscopy suites and clinical offices where the anesthesia machines may not be available. Non-limiting examples of trial uses of the nasal mask assembly 10 are provided below.

### Example 1

An obese patient with Class IV airway presented for laparoscopy. The patient was well preoxygenated with a face mask in a beach chair position (20 degree incline) and two pillows under her shoulders. After general anesthesia was induced, an operator could not obtain adequate face-mask seal with both small hands in order for the anesthesia attending to deliver assisted ventilation without or with oral airway. The attending could ventilate the patient alone with oral airway.

After removing the oral airway, a nasal mask assembly 10 with fully inflated air cushion was secured over her nose with head straps and connected to an adult breathing circuit. The attendant then closed the patient's mouth and obtained tight nose-mask seal with the left hand and easily ventilated the patient with the right hand. Presence of capnography indicated successful assisted nasal ventilation. Endrotracheal intubation was easily performed using a videolaryngoscopy and the patient maintained 99-100% O₂ saturation throughout induction and intubation. She underwent uneventful laparoscopy without any complication. This simple technique using a nasal mask assembly 10 for nasal ventilation in an obese adult patient with difficult airway improved face-mask seal and assisted ventilation by the attendant during induction of general anesthesia.

### Example 2

A morbidly obese patient with IDDM, spina bifida, severe peripheral neuropathy, known difficult airway, prior failed endrotracheal intubation leading to emergency tracheostomy and severe OSA requiring home BiPAP presented for left below knee amputation. Two months prior, he tolerated ankle debridement without local anesthesia or sedation while breathing comfortably with the nasal mask assembly 10.

Hie received femoral/sciatic blocks with 2 mg midazolam. While lying on the OR table with 15 degree incline, he was breathing comfortably with the nasal mask assembly 10 secured over his nose with head straps and connected to anesthesia breathing circuit/machine with pressure-supported ventilation. However, he complained of pain with incision. After 50% nitrous oxide (N₂O) was added, he quickly became comfortable. Subsequent attempt to replace N₂O with air elicited pain. Hie again became very comfortable with 50% N₂O and maintained 100% O₂ saturation throughout with bi-level PAP. He tolerated the procedure well without any complication. The nasal mask assembly 10 is easy to assemble using existing anesthesia equipment and machine. It maintains spontaneous respiration and improves oxygenation in sedated obese patients with OSA. It can be used to deliver N₂O to supplement inadequate regional nerve block.

### Example 3

A patient presented with progressively increasing dyspnea, ascites, severe anemia and acute coronary syndrome. He had multiple co-morbidities including HiTN, NIDDM, CKD, hepatitis C, cirrhosis, esophageal varices, thrombocytopenia, coagulopathy, obesity (BMI 30 kg/m²) and OSA with “narrow upper airway”. He was scheduled for urgent EGD with capsule endoscopy. He was on nasal cannula (NC) O₂ (3 L/min) and O₂ saturation (Sat) was 99%. Previous anesthesia record revealed that his room air O₂ Sat was 99% and he tolerated well with deep propofol sedation during EGD one month prior. He was pre-oxygenated with NC O₂ 4 L/min and a TSE “Mask” 70 using a clean plastic sheet to cover his eyes, nose and mouth.

He was titrated to deep sedation with 10-20 mg propofol boluses (a total of 50 mg) and propofol infusion (150 mcg/kg/min). He maintained spontaneous respiration with 99% O₂ Sat.

Due to difficulty in inserting the endoscope, end tidal (ET) CO₂ tracing disappeared. It was suspected that his airway was obstructed. Jaw-thrust did not relieve the obstruction and his O₂ Sat dropped to 85%. The endoscope was removed and his nose was quickly covered with the nasal mask assembly connected to a breathing circuit and anesthesia. His O₂ Sat increased to 96% after 4 breaths of nasal mask ventilation with 6 L/min O₂ flow. He resumed spontaneous respiration (RR: 12-14 breaths/min, TV: 250-300 cc) with CPAP of 3-5 cm of H₂O (by adjusting the pressure-relief valve). He then received 30 mg bolus of propofol and propofl infusion rate was reduced (75 mcg/kg/min). FiO₂ was adjusted to 0.8 by reducing O₂ to 3 L/min and adding air (1 L/min). O₂ Sat was 100% throughout the rest of the procedure despite some difficulty with capsule insertion because of his narrow upper airway. He recovered from sedation quickly without problem. The nasal mask assembly improves oxygenation in OSA patients and can be used to proactively prevent severe desaturation.

### Example 4

An obese patient with hypertension, asthma, sarcoidosis and OSA presented for emergency incision and debridement of perirectal abscess/necrotizing fascitis in a weekend evening. Awake intubation was planned due to difficult airway. After albuterol nebulizer treatment in a beach chair position (45°), a nasal mask assembly 10 with fully inflated air cushion was secured over her nose with head straps and connected to anesthesia breathing circuit/machine with 6 L/min O₂ and 5-6 cm H₂O CPAP.

She received small doses of midazolam, fentanyl and propofol followed by Cetacaine spray and 2% lidocaine gel lollipop. However, O₂ saturation decreased from 100% to 80% due to gagging with lidocaine lollipop. Lollipop was immediately removed and assisted nasal ventilation was delivered (3 small breaths). She resumed spontaneous respiration and maintained 100% O₂ saturation. With additional 3% viscous lidocaine, she did not react to awake videolaryngoscopy which revealed her vocal cords easily. Endrotracheal tube was easily placed followed immediately with propofol and rocuronium. She tolerated the procedure well under general anesthesia and received respiratory support overnight until Post-Operative Day One. The nasal mask assembly 10 improved oxygenation in an obese OSA patient with difficult airway during awake intubation. It prevented severe desaturation by allowing immediate assisted nasal ventilation.

### Example 5

A patient with suspected OSA presented for supraventricular tachycardia (SVT) ablation under local anesthesia and “light sedation” at the request of cardiologist. After pre-oxygenation with nasal cannula O₂ 4 L/min and a simple face tent (TSE “Mask”) 70, she received 4 mg of midazolam and 75 mcg of fentanyl but was unable to tolerate catheter insertion under local anesthesia.

With propofol infusion (75 mcg/kg/min), she did not respond to further catheter insertion. However, her airway became obstructed and required jaw-thrust. A nasal mask assembly 10 with fully-inflated air cushion (10 cc air added) was secured over her nose with head straps and connected to anesthesia breathing circuit/machine. The adjustable pressure-limiting (APL) valve was adjusted to deliver 5-7 cm 1-120 CPAP with 4 L/min O₂ and 1 L/min air (0.6-0.8 FiO₂). The reservoir bag was inverted to provide great visual monitoring of respiration from the control room. She tolerated the procedure well with propofol infusion (75-80 mcg/kg/min) and maintained spontaneous respiration and 99-100% O₂ saturation O₂ throughout. The nasal mask assembly 10 maintained spontaneous respiration and improved oxygenation in a suspected OSA patient under deep sedation during SVT ablation. It also avoided frequent manipulation of an obstructed airway or insertion of nasal trumpet in a patient with heparinization.

### Example 6

A morbidly obese patient presented for gynecological examination under anesthesia, hysteroscopy and D&C for postmenopausal bleeding. She had multiple comorbidities including anemia, atrial fibrillation, COPD, CAD, CHF, HTN, Renal insufficiency, respiratory failure (required supplemental O₂ at nursing home), ambulatory difficulty (secondary to hip pain), lower extremity edema, gastroesophageal reflux, scoliosis, kyphosis, limited neck extension, a difficult airway and OSA requiring nocturnal CPAP. Because of her multiple comorbidities, it was decided to proceed with spinal anesthesia and minimal IV sedation.

After spinal anesthesia was performed, the patient assumed supine position, with her back elevated (15 degree incline), and a nasal mask assembly 10 was used and the APL valve was adjusted to deliver CPAP (6 cm H₂O) with a mixture (0.7 FiO₂) of O₂ (4 L/min) and air (1 L/min). She maintained spontaneous respiration with tidal volume of 167-200 ml, RR of 24-27 breaths/min and peak expiratory pressure of 12-14 cm HIO. Her O₂ saturation (Sat) was 100% throughout the procedure. She tolerated the procedure well and recovered without complication.

### Example 7

An obese patient with HTN, NIDDM, OSA and non-compliance with home CPAP, presented for atrial flutter ablation under sedation. A nasal mask assembly 10 was used to provide CPAP from the beginning. Sedation was initiated with 2 mg of midazolam and 50 mg of propofol. Remifentanyl, starting at 0.03 mcg/kg/min and increasing to 0.05 mcg/kg/min was used for improved procedure tolerance. Shortly after bolusing propofol, a one minute apneic episode was managed with assisted nasal ventilation. Once spontaneous respiration resumed, the APL, valve was adjusted to provide CPAP of 5-8 cm H₂O. Because the ETCO₂ waveforms were not adequately shown in an old capnography monitor and chest excursion was difficult to assess under the drapes, the reservoir breathing bag was inverted in order to exaggerate its movement and augment patient monitoring. The patient tolerated the procedure well without further episodes of apnea and maintained 99-100% O₂ Sat with 0.7-0.8 FiO₂ throughout.

### Example 8

A morbidly obese patient presented for irrigation and debridement of left ankle ulcer. He had prior extremely difficult endrotracheal intubation, status post tracheostomy several year prior, severe peripheral neuropathy, CAD, IDDM, spina bifida and OSA requiring home BiPAP (16/7 cm H₂O). Because of his multiple comorbidities and severe peripheral neuropathy, it was decided to proceed without local anesthesia or sedation. He was on nasal cannula (NC) O₂ (2 L/min) and his O₂ Sat was 95% while sitting upright. His oropharynx was pretreated with 5% lidocaine cream for possible awake fiber optic intubation if need arose.

He complained of dyspnea while lying down with a foam wedge (30 degree incline). His O₂ Sat decreased to 92% even with NC O₂ (5 L/min). He requested a BiPAP mask and was fit with an adult facial mask to provide CPAP using an adult anesthesia breathing circuit, but felt more comfortable when a nasal mask assembly 10 to provide CPAP (5 cm 1H₂O) with a mixture (0.75 FiO₂) of O₂ (5 L/min) and air (2 L/min). He maintained spontaneous respiration and 100% O₂ Sat throughout the procedure. He tolerated the procedure well without complication. The patient was happy with the nasal mask assembly 10.

### Example 9

An obese patient with OSA was scheduled for EGD with dilation of gastric outlet obstruction. He was NPO for 4 days since gastric banding. His room air O₂ saturation (Sat) was 94%.

The patient received a nasal mask assembly 10 which was secured with head straps. He breathed comfortably through an adult breathing circuit with the APL valve open and 10 L/min fresh O₂ flow. After pre-oxygenated with this nasal mask for 5 min, his O₂ Sat increased from 95% to 100%. A bite-block was then put in place and a nasal cannula was secured on the lower rim of the bite-block for CO₂ and O₂ monitoring. The APL valve was then adjusted to provide continuous flow of O₂ (6 L/min) and air (2 L/min) to provide about 0.8 FiO₂ and maintain CPAP of 2-5 cm of H₂O. The size of CO₂ wave form was increased after his mouth was covered with a clean, clear plastic sheet (TSE “Mask”) 70. Deep sedation was induced with small boluses of propofol (a total of 150 mg) and maintained with infusion (150 mcg/kg/min). He maintained spontaneous respiration and 99-100% O₂ saturation throughout. He tolerated the procedure well and recovered from sedation quickly.

### Example 10

A patient with ESRD presented to the ED for signs and symptoms of fluid overload following peritoneal dialysis. He was scheduled to have an AV fistula creation for permanent hemodialysis access. His comorbidities included HTN, CAD, anemia, obesity (BMI 35 kg/m²) and GERD. Although he never underwent polysomnography, he did admit to heavy snoring and had a large neck circumference making OSA highly likely.

After receiving midazolam and fentanyl, a supraclavicular nerve block was performed ultrasound guidance with 35 cc of 0.5% ropivicaine in the holding area. While in the OR, he received NC O₂ (4 L/min) and a face tent (TSE “Mask”) 70. His O₂ saturation (Sat) was 96% prior to sedation. He then received 30 mg lidocaine and 50 mg propofol and propofol infusion (100-150 mcg/kg/min). Soon after propofol infusion, his airway was obstructed as indicated by losing the ETCO₂ tracing. He required constant chin lift and jaw thrust to maintain patient airway. Rather than continuing these manipulations for the duration of the case, a fully inflated nasal mask assembly 10 was secured over his nose with head straps and connected to a breathing circuit and anesthesia machine. The APL valve was adjusted to deliver CPAP of 8 cm 1-120 with 4 L/min O₂ and 1 L/min air yielding 0.7-0.8 FiO₂. After the nasal mask/circuit was secured, he resumed spontaneous respiration as indicated by ETCO₂. The patient maintained spontaneous respirations (TV 250-300 cc and RR 12-20 breaths/min) and 100% O₂ Sat throughout the remainder of the case and emerged from sedation uneventfully. He was discharged home later that evening after meeting appropriate discharge criteria. The nasal mask assembly 10 maintained spontaneous respiration and oxygenation in an obese patient with unrecognized OSA under intraoperative sedation.

### Example 11

A patient with lung cancer, obesity, severe asthma, OSA, NIDDM, HTN, CAD and GERD presented for radiotherapy of RUL lung cancer. Sedation was needed because of required abdominal compression during treatment. After albuterol nebulizer treatment, her room air O₂ saturation (Sat) was 88-91%. She was pretreated with 10 mg metoclopramide 45 min before the procedure to decrease the risk of aspiration due to abdominal compression. Her O₂ Sat increased to 94% with nasal cannula O₂ (4 L/min) and 100% by adding a simple face tent (TSE “mask”) 70. However, she became apneic with small boluses of propofol (2×30 mg) and her O₂ Sat decreased quickly to 80%. A nasal mask assembly 10 with a fully-inflated air cushion was placed over her nose and connected to anesthesia breathing circuit/machine with 8 L/min O₂. Her O₂ Sat increased to 100% with assisted nasal ventilation (4-5 breaths). She resumed spontaneous respiration with 12-14 cm HO CPAP with 4 I/min O₂ and 1 L/min air. She tolerated the abdominal compression well with propofol infusion (50-75 mcg/kg/min) and maintained 100% O₂ Sat (0.8 FiO₂) throughout. The reservoir bag was inverted to provide great visual monitoring of respiration from the control room. She tolerated the radiotherapy several times very well with the nasal mask assembly and required a nasal trumpet to keep her nasal airway open when a full face mask was used. The nasal mask/circuit assembly maintained spontaneous respiration and oxygenation in an obese patient with OSA and severe pulmonary disease during repeated radiotherapies with abdominal compression.

### Example 12

A patient presented for screening colonoscopy. He had OSA and used a nocturnal home CPAP. He had two previous upper GI endoscopic procedures under deep propofol sedation. His oxygenation was well maintained with a TSE “Mask” 70. However, he required frequent jaw-thrust to maintain a patent airway. His room air Oz saturation (Sat) was 96% while sitting up and decreased to 94% while lying down on 2 pillows. His O₂ Sat increased to 98% with NC O₂ (4 L/min).

A nasal mask assembly 10 was secured and connected to an adult breathing circuit attached to the anesthesia machine. The APL valve adjusted to deliver CPAP of 5-7 cm H₂O with mixture (0.8 FiO₂) of O₂ (4.5 L/min) and air (1.5 L/min). After pre-oxygenated using the nasal mask/circuit for 2-3 min, his O₂ Sat increased to 100%/o. He received 100 mg lidocaine and was then titrated to deep sedation with two 50 mg propofol boluses and propofol infusion (150 mcg/kg/min). He maintained spontaneous respiration (RR: 14-17 breaths/min, TV: 200-250 cc, Peak Expiratory Pressure: 12-15 cm H₂O, ETCO₂: 22-42 mm Hg) with 100% O₂ Sat throughout the procedure. A few min after stopping propofol infusion, the nasal mask seal was reduced by adjusting the head-straps tension. ETCO₂ disappeared and CPAP was zero. It indicated that his airway was obstructed and he became apneic. After head straps were re-adjusted to obtain a tight seal, CPAP increased immediately to 5 cm H₂O and he resumed spontaneous respiration as indicated by returning of ETCO₂ tracing and movement of the reservoir bag. The nasal mask assembly 10 was replaced with NC O₂ after he was awake. The patient recovered quickly and his room air O₂ Sat was 98% prior to discharge home.

This nasal mask assembly 10 maintained spontaneous respiration and improved oxygenation in all obese OSA patients of Examples 2-12 during intraoperative sedation or awake intubation. It proactively prevented severe desaturation by allowing immediate assisted nasal mask ventilation.

### Example 13

A child patient presented for elective bronchoscopy with bronchioalveolar lavage to rule out possible etiology of recurrent pneumonias. He was an ex-premature twin born via cesarean section with no significant birth complications. He has poorly controlled asthma triggered by environmental exposure to cold and upper respiratory tract infections (URI). Parents are active tobacco smokers with a new cat in the home. He takes montelukast and routine albuterol and fluticasone aerosols. There have been multiple hospitalizations for pneumonia, but no URIS, pneumonias, or asthma exacerbations within the past 6 weeks of procedure. The lungs are clear on auscultation as well as CXR and the immune work up done was normal.

After an inhalational induction with a standard face mask with N₂O 70%, O₂ 30%, and 8% sevoflurance, a size 2 laryngeal mask airway (LMA) was placed. Propotol, and fentanyl, and decadron were given. A deep plane of anesthesia was obtained with sevoflurane before a flexible pediatric bronchoscope was inserted via LMA. Bronchoscopy showed a normal bronchial anatomy and secretions in the right lower lobe. Following lung lavage with normal saline, the patient experienced laryngospasm with desaturation to 85-90% with no end tidal CO₂. The bronchoscope was removed and succinylcholine 10 mg IV and epinephrine 2 mcg IV given, and O₂ Sat improved >97%. Shortly after improvement in O₂ Sat, the patient was found to have bilateral wheezes and coarse breath sounds over the right chest. Albuterol MDI was administered via LMA and sevotflurance increased to 4%. Additional decadron was given (total dose 0.5 mg/kg). After improvement in O₂ Sat and decreased wheezing, LMA was removed and the oral airway was placed under deep plane of sevoflurane anesthesia. The patient became stridorous and wheezing was heard again predominantly over the right chest. No ETCO₂ was present. Jaw thrust and CPAP with a standard size face mask were attempted with improvement of O₂ Sat to 90%.

A nasal mask assembly 10, in this case in the form of a neonatal mask, with air cushion inflated with 4 ml of air was secured over the patient's nose. The APL valve was closed to give CPAP. Nebulizer mask was placed over the mouth to give racemic epinephrine and albuterol treatments with the nasal CPAP. After 10 minutes of nasal CPAP and nebulized treatments, O₂ Sat improved to greater than 95%. The patient was transported to the post anesthesia care unit. One hour later, another racemic epinephrine treatment was given. The patient was sent to PICU for monitoring overnight and discharged home in stable condition. The use of the nasal mask assembly 10 can be equally beneficial for children. Nasal CPAP using the nasal mask assembly 10 while simultaneously orally administering nebulized bronchodilator and racemic in an asthmatic toddler patient with laryngospasm and wheezing was effective.

These and other advantages of the present invention will be apparent to those skilled in the art from the foregoing specification. Accordingly, it will be recognized by those skilled in the art that changes or modifications may be made to the above-described embodiments without departing from the broad inventive concepts of the invention. It should therefore be understood that this invention is not limited to the particular embodiments described herein, but is intended to include all changes and modifications that are within the scope and spirit of the invention as defined in the claims.

What is claimed:

1. A nasal mask assembly for use with a patient, the nasal mask assembly including:
a nasal mask with a dome shaped body having an inner rim and an outer surface with a tubing connector extending from the outer surface, wherein the inner rim defines a perimeter which is configured such that a bottom portion of the rim will sit on or above the patient's upper lip while an upper portion of the rim will extend across a bridge of the patient's nose; and
a securing assembly for securing the nasal mask on the patient.

2. The nasal mask assembly according to claim 1 further comprising an inflatable chamber extending about the inner rim with a sealed inflation port proximate the bottom portion of the rim.

3. The nasal mask assembly according to claim 1 wherein the securing assembly includes a plurality of flexible straps with holes configured to engage pegs extending from the nasal mask.

4. The nasal mask assembly according to claim 3 wherein the connection pegs extend from a connection ring positioned about the tubing connector.

5. The nasal mask assembly according to claim 1 further comprising a bite block configured to be positioned in the patient's mouth while the nasal mask is secured over the patient's nose.

6. The nasal mask assembly according to claim 5 further comprising a nasal cannula secured proximate to the patient's open mouth, the nasal cannula configured to intake CO₂ and O₂ proximate to the patient's mouth.

7. The nasal mask assembly according to claim 6 further comprising a connector for connecting a sampling line of the nasal mask and the nasal cannula to a monitoring system to monitor the CO₂ and O₂ level.

8. The nasal mask assembly according to claim 1 wherein in an adult patient, the nasal mask has the size of a standard pediatric mask.

9. The nasal mask assembly according to claim 1 wherein in a child patient, the nasal mask has the size of a standard neonatal mask.

10. A method of treating a patient comprising the steps of:
securing a nasal mask over a face of the patient such that that a bottom portion of the rim sits on or above the patient's upper lip while an upper portion of the rim extends across a bridge of the patient's nose while leaving the patient's mouth uncovered by the nasal mask;
administering a fluid to the patient through the nasal mask.

11. The method according to claim 10 wherein the securing step includes attaching straps about the patient's head and securing them to the nasal mask.

12. The method according to claim 11 wherein the securing step further comprises inflating an inflatable chamber extending about the inner rim.

13. The method according to claim 10 further comprising the step of positioning a treatment tool in the patient's mouth unencumbered by the nasal mask.

14. The method according to claim 13 wherein the treatment tool includes a bite blocker.

15. The method according to claim 10 further comprising the step of monitoring the CO₂ and O₂ levels proximate to the patient's mouth unencumbered by the nasal mask.
